(19) United States
(12) Patent Application Publication
(10) Pub. No.: US 2015/0087041 A1
(43) Pub. Date: Mar. 26, 2015

(54) PRODUCTION OF ETHANOL WITH REDUCED CONTAMINANTS IN A CELLULOSIC BIOMASS BASED PROCESS

(71) Applicant: E I DU PONT DE NEMOURS AND COMPANY, Wilmington, DE (US)

(72) Inventor: WILLIAM D. PARTEN, Wilmington, DE (US)

(73) Assignee: E I DU PONT DE NEMOURS AND COMPANY, Wilmington, DE (US)

(21) Appl. No.: 14/037,411

(22) Filed: Sep. 26, 2013

(51) Int. Cl. C12P 7/10

(52) U.S. Cl. CPC C12P 7/10
USPC 435/165

(57) ABSTRACT

Processes for producing ethanol from fermentation systems employing cellulosic feedstocks are provided, where contaminants prevalent from biomass hydrolysate fermentation are managed. The management of contaminants allows recycle of process water between the fermenter vent scrubber and rectification column, thereby removing the need for fresh water to the scrubber.

### FIELD OF THE INVENTION

The present invention relates to processes for producing ethanol in a fermentative process from cellulosic feedstocks. Specifically, contaminants prominent in ethanol produced using biomass hydrolysate are removed from the final product, which also allows recycle of process water.

### BACKGROUND OF THE INVENTION

Ethanol is an important source of energy and useful as an alternative to petroleum based gasoline and diesel products. Ethanol is produced by fermentation of a wide variety of organic feedstocks to provide a beer that is distilled and dehydrated to produce a high purity product. The majority of fuel ethanol today is produced from grain, starch or sugar based feedstocks. These methods typically include fermentation of a mixture of water and milled grain to yield alcohol, distillation of the fermented mixture to recover alcohol as a top product and distillery bottom by-products, which includes grain solids and thin stillage of dissolved solids in water. The distillary by-products are typically concentrated by evaporation of water therefrom, to yield Distiller's Dried Grains with Solubles (DDGS), a valuable feed for livestock.

Typical grain ethanol facilities generally have the following elements in common:
1) There is an absorption or scrubbing process on the vent stream from the fermenters to recover methanol, ethanol, higher alcohols and acetaldehyde from the co-produced carbon dioxide. This normally draws the makeup water into the plant and is typically a cold stream.
2) Distillation is employed to produce a stream that is concentrated in ethanol up to the azeotropic composition with water and a predominantly water and solids stream that is free of ethanol. This process is generally achieved by use of two distillation columns known as a Beer Column and a Rectifying Column
3) The azeotropic ethanol stream is then subject to further concentration via a two liquid phase azeotropic distillation involving an entrainer or more usually by concentration in molecular sieves to produce a fuel grade ethanol which is the prime product of the process.
4) The solids from the aqueous stream mentioned in 2) are separated via centrifugation or some other means and may be further dried to produce an animal feed or material for fuel.
5) A fraction of the aqueous stream (backset) resulting from 4) may be recycled to the front end of the plant to form a fraction of the feed to the fermenter whilst the remainder will be evaporated to remove impurities that would otherwise build up. These impurities include sugars that can't be fermented, proteins and salts and are purged as a concentrated liquid stream. The condensate from the evaporation may be recycled directly to the fermentation with the backset. The evaporation process is usually two or three stages and will be heat integrated with the distillation process.

Grain, starch and sugar based processes are becoming increasingly less desirable as they necessarily rely on a food source and have negatively impacted global food prices. Production of ethanol from cellulosic agricultural and other waste feedstocks avoids these problems. Cellulosic feedstocks are those that typically contain cellulose and hemi-cellulose, as well as lignin. Suitable feedstocks for the production ethanol from cellulosic feedstocks include biomass such as corn cob, corn stover, grasses, woody biomass, sugar cane bagasse, as well as industrial waste products containing a high cellulosic component. Processes for the generation of alcohols and particularly ethanol from cellulosic feedstocks are described in numerous publications (see for example Aden et al. in “Lignocellulosic Biomass to Ethanol Process Design and Economics Utilizing Co-Current Dilute Acid Prehydrolysis and Enzymatic Hydrolysis for Corn Stover”, NREL Report No. TP-510-32438; NREL Report NREL/TP-510-37092, “Plants from Corn Starch and Lignocellulosic Feedstocks (Revised)”; and Madson, P. W. and D. A. Monceaux, (Fuel Ethanol Production), in “Fuel Alcohol Textbook” Alltech Inc., 1999. Various aspects of a cellulosic ethanol process are disclosed in commonly owned patents U.S. Pat. No. 7,629,156, U.S. Pat. No. 7,666,282 U.S. Pat. No. 7,741,084, U.S. Pat. No. 7,741,119, U.S. Pat. No. 7,781,191, U.S. Pat. No. 7,803,623, U.S. Pat. No. 7,807,419, U.S. Pat. No. 7,819,976, U.S. Pat. No. 7,897,396, U.S. Pat. No. 7,910,338, U.S. Pat. No. 7,932,063, U.S. Pat. No. 7,989,206, U.S. Pat. No. 7,998,713, U.S. Pat. No. 7,998,722, U.S. Pat. No. 8,216,809, U.S. Pat. No. 8,241,873, U.S. Pat. No. 8,241,880, U.S. Pat. No. 8,247,208, U.S. Pat. No. 8,278,070, U.S. Pat. No. 8,304,213, and U.S. Pat. No. 8,304,535.

Processes for cellulosic biomass production of alcohols face certain challenges not present in typical grain ethanol plants. A major challenge is an increased level of contaminants that co-separate with the ethanol, as compared to contaminant levels in a grain ethanol process. In particular, acetaldehyde forms a major contaminant in the ethanol product when biomass hydrolysate is a major component of the fermentation medium. Ethanol produced in a grain based process typically contains from about 200 to 500 ppm of acetaldehyde, while ethanol produced using biomass hydrolysate typically contains higher levels. Considering the hazardous nature of acetaldehyde and its volatility that will exert a significant vapor pressure above a gasoline blend, target levels in a cellulosic ethanol product may be similar to those found in fuel ethanol from a grain process. In addition, when ammonia is used for biomass pretreatment prior to saccharification producing the hydrolysate, ammonia is another potential contaminant in the ethanol product.

In addition, a cellulosic ethanol process has increased water consumption as compared to a grain ethanol process due to the reduced ethanol concentration in the fermentation broth, which is typically in the range of 5% to 10% ethanol vs 11% to 15% ethanol for grain based ethanol production. Thus recycle of water is important in a cellulosic ethanol process to limit the amount of fresh makeup water brought into the process.

Thus simply transferring the methods of working that are established within the grain ethanol industry to a cellulosic ethanol process is not sufficient for economical production of a comparable ethanol product. Hence cellulosic ethanol presents a new set of challenges, and processes that are able to meet those challenges should be considered against that background rather than in the context of a grain based process.

There remains a need to develop an effective process for production of ethanol using biomass hydrolysate fermentation that effectively processes contaminants such as acetaldehyde, producing an ethanol product low in contaminants prevalent in the cellulosic process. In addition, recycle of water streams is desired to reduce fresh water usage.

### SUMMARY OF THE INVENTION

Production of fuel ethanol from cellulosic fermentations faces challenges in ethanol contaminants and in water usage not seen in comparable grain ethanol processes. In order to remove ethanol contaminants, applicants provide a system with a further ethanol purification step. The process is made more efficient by recycle of process water streams, thereby reducing input of fresh makeup water.

Accordingly the invention provides a process for the production of ethanol comprising:
a) providing medium comprising hydrolysate prepared from cellulosic biomass;
b) fermenting the medium in a fermenter in the presence of a microorganism that produces ethanol to produce a beer comprising ethanol;
c) passing the beer into a beer column where a beer column ethanol-rich vapor stream is produced;
d) condensing the beer column ethanol-rich vapor stream forming a beer column ethanol-rich stream;
e) passing the beer column ethanol-rich stream into a rectification column where a further ethanol-enriched rectification column vapor stream and an ethanol depleted rectification column water stream are produced;
f) passing the further ethanol-enriched rectification column vapor stream through a molecular sieve where a molecular sieve ethanol product stream is produced; and
g) passing the molecular sieve ethanol product stream through a product distillation column where a final ethanol product and a contaminant stream are produced.

In one embodiment the final ethanol product has less than about 800 ppm of acetaldehyde.

In one embodiment the process further comprises:
h) passing at least a portion of the ethanol depleted rectification column water stream of (e) to a fermentation vapor scrubber that is connected to a vent stream from the fermenter, producing a scrubber water stream comprising water, alcohol, acetaldehyde, and carbon dioxide; and
i) passing the scrubber water stream to the rectification column; wherein ethanol depleted rectification column water and fermentation vapor scrubber water form a water recycle loop between the rectification column and fermentation vapor scrubber.

### BRIEF DESCRIPTION OF THE FIGURES

FIG. 1 is a schematic diagram of a cellulosic ethanol process flow sheet.

### DETAILED DESCRIPTION OF THE INVENTION

The following definitions and abbreviations are to be use for the interpretation of the claims and the specification.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers. For example, a composition, a mixture, a process, a method, an article, or an apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “consists of,” or variations such as “consist of” or “consisting of,” as used throughout the specification and claims, indicate the inclusion of any recited integer or group of integers, but that no additional integer or group of integers may be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

Also, the indefinite articles “a” and “an” preceding an element or component of the invention are intended to be nonrestrictive regarding the number of instances, i.e., occurrences of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the application.

As used herein, the term “about” modifying the quantity of an ingredient or reactant of the invention employed refers to variation in the numerical quantity that can occur, for example, through typical measuring and liquid handling procedures used for making concentrates or solutions in the real world; through inadvertent error in these procedures; through differences in the manufacture, source, or purity of the ingredients employed to make the compositions or to carry out the methods; and the like. The term “about” also encompasses amounts that differ due to different equilibrium conditions for a composition resulting from a particular initial mixture. Whether or not modified by the term “about”, the claims include equivalents to the quantities. In one embodiment, the term “about” means within 10% of the reported numerical value, preferably within 5% of the reported numerical value.

“Stripping” as used herein means the action of transferring all or part of a volatile component from a liquid stream into a gaseous stream.

“Scrubbing” or “scrubber” refers to a device or system that can removes particles, gases or other contaminants from an industrial process. Scrubber systems as used herein may use recycled water streams to remove CO₂ from fermenter streams.

“Rectifying” as used herein means the action of transferring all or part of a condensable component from a gaseous stream into a liquid stream in order to separate and purify lower boiling point components from higher boiling point components.

The term “lignocellulosic” refers to a composition comprising both lignin and cellulose. Lignocellulosic material may also comprise hemicellulose.

The term “cellulosic” refers to a composition comprising cellulose and additional components, including hemicellulose. A cellulosic composition may also include lignin.

The term “saccharification” refers to the production of fermentable sugars from polysaccharides.

The term “fermentable sugar” refers to oligosaccharides and monosaccharides that can be used as a carbon source by a microorganism in a fermentation process.

The term “pretreated biomass” means biomass that has been subjected to pretreatment prior to saccharification.

The term “lignocellulosic biomass” refers to any lignocellulosic material and includes materials comprising cellulose, hemicellulose, lignin, starch, oligosaccharides and/or monosaccharides. Biomass may also comprise additional components, such as protein and/or lipid. Biomass may be derived from a single source, or biomass can comprise a mixture derived from more than one source; for example, biomass could comprise a mixture of corn cobs and corn stover, or a mixture of grass and leaves. Lignocellulosic biomass includes, but is not limited to, bioenergy crops, agricultural residues, municipal solid waste, industrial solid waste, sludge from paper manufacture, yard waste, wood and forestry waste. Examples of biomass include, but are not limited to, corn cobs, crop residues such as corn husks, corn stover, grasses, wheat straw, barley straw, hay, rice straw, switchgrass, waste paper, sugar cane bagasse, sorghum plant material, soybean plant material, components obtained from milling of grains, trees, branches, roots, leaves, wood chips, sawdust, shrubs and bushes, vegetables, fruits, and flowers.

The term “hydrolysate” refers to the product resulting from saccharification of biomass. The biomass may also be pretreated or pre-processed prior to saccharification.

The term “biomass hydrolysate fermentation broth” is broth containing product resulting from biocatalyst growth and production in a medium comprising biomass hydrolysate. This broth includes components of biomass hydrolysate that are not consumed by the biocatalyst, as well as the biocatalyst itself and product made by the biocatalyst.

The present process provides for ethanol production from cellulosic biomass including further purification of the product that corresponds to the ethanol product from a grain ethanol process. The further purification is used to remove contaminants that occur due to the use of hydrolysate in the fermentation medium. This further purification allows process water streams to be recycled, providing efficiency of water use including reduced makeup water input in the cellulosic ethanol process.

### Biomass Hydrolysate

The present process is a cellulosic ethanol process in which medium used in fermentation contains hydrolysate prepared from cellulosic biomass, which is a hydrolysate fermentation medium. The biomass used may be any cellulosic or lignocellulosic material, for example, bioenergy crops, agricultural residues, municipal solid waste, industrial solid waste, yard waste, wood, forestry waste and combinations thereof. Cellulosic biomass hydrolysate is produced by saccharification of cellulosic (including lignocellulosic) biomass. Typically the biomass is pretreated prior to saccharification. Biomass may be treated by any method known by one skilled in the art to produce fermentable sugars in a hydrolysate. Typically the biomass is pretreated using physical and/or chemical treatments, and saccharified enzymatically. Physical and chemical treatments may include grinding, milling, cutting, base treatment such as with ammonia or NaOH, and/or acid treatment. Particularly useful is a low ammonia pretreatment where biomass is contacted with an aqueous solution comprising ammonia to form a biomass-aqueous ammonia mixture where the ammonia concentration is sufficient to maintain an alkaline pH of the biomass-aqueous ammonia mixture but is less than about 12 wt. % relative to dry weight of biomass, and where dry weight of biomass is at least about 15 wt % solids relative to the weight of the biomass-aqueous ammonia mixture, as disclosed in commonly owned U.S. Pat. No. 7,932,063, which is herein incorporated by reference.

Enzymatic saccharification of cellulosic or lignocellulosic biomass typically makes use of an enzyme composition or blend to break down cellulose and/or hemicellulose and to produce a hydrolysate containing sugars such as, for example, glucose, xylose, and arabinose. Saccharification enzymes are reviewed in Lynd, L. R., et al. (Microbiol. Mol. Biol. Rev., 66:506-577, 2002). At least one enzyme is used, and typically a saccharification enzyme blend is used that includes one or more glycosidases. Glycosidases hydrolyze the ether linkages of di-, oligo-, and polysaccharides and are found in the enzyme classification EC 3.2.1.x (Enzyme Nomenclature 1992, Academic Press, San Diego, Calif. with Supplement 1 (1993), Supplement 2 (1994), Supplement 3 (1995, Supplement 4 (1997) and Supplement 5 [in Eur. J. Biochem., 223:1-5, 1994; Eur. J. Biochem., 232:1-6, 1995; Eur. J. Biochem., 237:1-5, 1996; Eur. J. Biochem., 250:1-6, 1997; and Eur. J. Biochem., 264:610-650 1999, respectively]) of the general group “hydrolases” (EC 3.). Glycosidases useful in the present method can be categorized by the biomass components they hydrolyze. Glycosidases useful for the present method include cellulose-hydrolyzing glycosidases (for example, cellulases, endoglucanases, exoglucanases, cellobiohydrolases, β-glucosidases), hemicellulose-hydrolyzing glycosidases (for example, xylanases, endoxylanases, exoxylanases, β-xylosidases, arabino-xylanases, mannases, galactases, pectinases, glucuronidases), and starch-hydrolyzing glycosidases (for example, amylases, α-amylases, β-amylases, glucoamylases, α-glucosidases, isoamylases). In addition, it may be useful to add other activities to the saccharification enzyme consortium such as peptidases (EC 3.4.x.y), lipases (EC 3.1.1.x and 3.1.4.x), ligninases (EC 1.11.1.x), or feruloyl esterases (EC 3.1.1.73) to promote the release of polysaccharides from other components of the biomass. It is known in the art that microorganisms that produce polysaccharide-hydrolyzing enzymes often exhibit an activity, such as a capacity to degrade cellulose, which is catalyzed by several enzymes or a group of enzymes having different substrate specificities. Thus, a “cellulase” from a microorganism may comprise a group of enzymes, one or more or all of which may contribute to the cellulose-degrading activity. Commercial or non-commercial enzyme preparations, such as cellulase, may comprise numerous enzymes depending on the purification scheme utilized to obtain the enzyme. Many glycosyl hydrolase enzymes and compositions thereof that are useful for saccharification are disclosed in WO 2011/038019. Additional enzymes for saccharification include, for example, glycosyl hydrolases that hydrolyze the glycosidic bond between two or more carbohydrates, or between a carbohydrate and a noncarbohydrate moiety.

Saccharification enzymes may be obtained commercially. Such enzymes include, for example, Spezyme® CP cellulase, Multifect® xylanase, Accelerase® 1500, and Accellerase® DUET (Danisco U.S. Inc., Genencor International, Rochester, N.Y.), and Novosyme-188 (Novozymes, 2880 Bagsvaerd, Denmark). In addition, saccharification enzymes may be unpurified and provided as a cell extract or a whole cell preparation. The enzymes may be produced using recombinant microorganisms that have been engineered to express one or more saccharifying enzymes. For example, the H3A protein preparation used herein for saccharification of pretreated cellulosic biomass is an unpurified preparation of enzymes produced by a genetically engineered strain of Trichoderma reesei, which includes a combination of cellulases and hemicellulases and is described in WO 2011/038019, which is incorporated herein by reference.

Fermentation media containing biomass hydrolysate may contain a percent of hydrolysate with one or more additional sugars and/or other added components, or the media may contain 90% or more hydrolysate with minor additions. To improve growth, sorbitol, mannitol, or a mixture thereof may be included in the medium as disclosed in commonly owned U.S. Pat. No. 7,629,156, which is incorporated herein by reference. Typically a final concentration of about 5 mM sorbitol or mannitol is present in the medium. In various embodiments cellulosic biomass hydrolysate is at least about 50%, 60%, 70%, 80%, 90% or 95% of the final volume of fermentation broth. Typically about 10% of the final volume of fermentation broth is seed inoculum containing the biocatalyst.

The solids content of biomass hydrolysate is typically between about 10% and 40%, depending on the pretreatment and saccharification methods employed. More typically the solids content is about 25%, with a medium containing 90% cellulosic biomass hydrolysate having about 23% solids.

### Fermentation

In the present process the medium comprising hydrolysate is fermented in a fermenter, which is any vessel that holds the hydrolysate fermentation medium and biocatalyst, and has valves, vents, and/or ports used in managing the fermentation process. In the present process the biocatalyst is a microorganism that produces ethanol. The microorganism may naturally produce ethanol, or be genetically engineered to produce ethanol, or to have improved ethanol production. Any of these microorganisms is an ethanologen. In one embodiment the ethanologen is a yeast or a bacterium. In one embodiment the yeast is of the genus Saccharomyces. In one embodiment the bacterium is of the genus Zymomonas or Zymobacter.

The biocatalyst may be engineered to have improved ethanol production in hydrolysate medium. The biocatalyst may be engineered for xylose utilization such as in Saccharomyces cerevisiae which is described in Matsushika et al. (Appl. Microbiol. Biotechnol. (2009) 84:37-53) and in Kuyper et al. (FEMS Yeast Res. (2005) 5:399-409). The biocatalyst may be engineered for xylose utilization such as in Zymomonas mobilis which is described in U.S. Pat. No. 5,514,583, U.S. Pat. No. 5,712,133, U.S. Pat. No. 6,566,107, WO 95/28476, Feldmann et al. ((1992) Appl Microbiol Biotechnol 38: 354-361), and Zhang et al. ((1995) Science 267:240-243). Examples of strains engineered to express a xylose utilization metabolic pathway include CP4(pZB5) (U.S. Pat. No. 5,514,583), ATCC31821/pZB5 (U.S. Pat. No. 6,566,107), 8b (US 20030162271; Mohagheghi et al., (2004) Biotechnol. Lett. 25; 321-325), and ZW658 (ATTCC # PTA-7858). The biocatalyst may be engineered for arabinose utilization as described in U.S. Pat. No. 5,843,760, and US 2011/0143408, which are incorporated herein by reference. Examples of additional modifications that may be engineered in Zymomonas include reduced expression of the endogenous himA gene as described in U.S. Pat. No. 7,897,396, which is incorporated herein by reference; reduced glucose-fructose oxidoreductase (GFOR) activity as described in U.S. Pat. No. 7,741,119, which is incorporated herein by reference; increased ribose-5-phosphate isomerase (RPI) activity, as disclosed in commonly owned and co-pending US 20120156746, which is incorporated herein by reference; expression of xylose isomerase as part of the xylose utilization metabolic pathway using a mutant, highly active promoter as disclosed in U.S. Pat. No. 7,989,206 and U.S. Pat. No. 7,998,722, which are incorporated herein by reference; expression of a Group I xylose isomerase as disclosed in commonly owned and co-pending US 2011-0318801, which is incorporated herein by reference; and adaptation of a strain for growth in stress culture containing ethanol and ammonium acetate as disclosed in US 2011-0014670, which is incorporated herein by reference.

Fermentation is carried out with conditions appropriate for the particular biocatalyst used. Adjustments may be made for conditions such as pH, temperature, oxygen content, and mixing. Conditions for fermentation of yeast and bacterial biocatalysts are well known in the art.

In addition, saccharificatoin and fermentation may occur at the same time in the same vessel, called simultaneous saccharification and fermentation (SSF). In addition, partial saccharification may occur prior to a period of concurrent saccharification and fermentation in a process called HSF (hybrid saccharification and fermentation).

For large scale fermentations, typically a smaller culture of the biocatalyst is first grown, which is called a seed culture. The seed culture is added to the fermentation medium as an inoculum typically in the range from about 2% to about 20% of the final volume.

Typically fermentation by the biocatalyst produces a beer containing from about 6% to about 10% ethanol. The beer may contain between about 7% and about 9% of ethanol. In addition, the beer contains water, solutes, and solids from the hydrolysate medium and from biocatalyst metabolism of sugars in the hydrolysate medium. In particular, the beer contains acetaldehyde in levels that are higher than those found in a beer produced from grain fermentation. In addition, when ammonia is used for pretreatment of the biomass prior to saccharification producing hydrolysate used in fermentation media, ammonia is present in the beer. These contaminants have high volatility and will co-purify with the ethanol product during distillation.

### Ethanol Purification

Beer produced from biomass hydrolysate fermentation, which contains ethanol, water, solutes, and solids, is passed to a beer column where an ethanol-rich vapor stream is separated from a water stream containing solutes and solids, also called whole stillage. Typically solids are separated from the beer column water stream using a filter press, centrifugation, or other solid separation method. The remaining water containing solutes, also called thin stillage, is passed through an evaporation train to produce a syrup, containing low-volatility solutes, and water vapor, containing high-volatility solutes, that may be condensed and further treated to remove contaminants, then recycled. Treatment may be using an anaerobic digester. Use of anaerobic digesters is well known by one skilled in the art for bacterial hydrolysis of organic materials, and typically production of methane and carbon dioxide. This biogas may be used directly as fuel, or upgraded to higher quality biomethane fuel. The evaporation train is described further below.

The beer column ethanol-rich vapor stream is typically about 30%-55% ethanol by volume. The ethanol-rich vapor stream is condensed and passed to a rectification column where a further ethanol-enriched rectification column vapor stream is produced, as well as an ethanol depleted water stream. The further ethanol-enriched rectification column vapor stream is typically about 90 to 95% ethanol by volume, which is close to the ethanol/water azeotrope (95.63% ethanol and 4.37% water, by weight). This stream is super-heated and passed to a molecular sieve for further water removal producing a molecular sieve ethanol product. This ethanol product is about 99% ethanol by volume.

The condensed molecular sieve ethanol product is typically the final ethanol product in a grain ethanol process. The corresponding molecular sieve ethanol product in a cellulosic ethanol process, where biomass hydrolysate is fermented, contains levels of contaminants not found in the grain ethanol product. Management of these contaminants needs to be addressed in the cellulosic ethanol process. Specifically, applicants have measured acetaldehyde in the molecular sieve ethanol product from a hydrolysate fermentation process and found the level to be higher than the 200 to 500 ppm typically found in a grain ethanol molecular sieve ethanol product.

In the present process the molecular sieve ethanol product is passed through a product distillation column. Distillation in this column is carried out so that acetaldehyde, ammonia and carbon dioxide are concentrated overhead, and the bottoms stream is the final ethanol product. The operating pressure of the distillation column may be linked to that of the molecular sieve unit so that material flows to the column by pressure difference. The operating pressure may also be high enough so that reflux can be returned to the column by use of a condenser utilizing cooling water for heat removal, such that there are minimal ethanol losses overhead. The overheads ethanol composition may be less than 50%, less than 30% or preferably less than 15% ethanol. The molecular sieve alcohol product stream may be passed through a condenser or partial condenser prior to passing it through the product distillation column.

The ethanol product from the product distillation column is the final ethanol product. This product contains reduced levels of acetaldehyde in comparison to the molecular sieve ethanol product. In one embodiment the final alcohol product contains less than about 800 ppm of acetaldehyde. In various embodiments the final alcohol product contains less than 800 ppm, 700 ppm, 600 ppm, 500 ppm, 400 ppm or 300 ppm of acetaldehyde. In addition, the final ethanol product typically contains reduced levels of other contaminants such as carbon dioxide and ammonia, in comparison to the molecular sieve ethanol product. Typically the final ethanol product contains less than about 10 ppm of CO₂, and less than about 1 ppm of ammonia.

A contaminant stream is produced from the product distillation column. This stream is treated to avoid release of acetaldehyde and other contaminants to the atmosphere. The stream may be treated by any method known by one skilled in the art for removing the contaminants, such as acetaldehyde, CO₂, and/or ammonia. In various embodiments the product distillation column contaminant stream is treated in a boiler, a catalytic converter, a catalytic oxidizer, a thermal oxidizer, or in any combination of these units.

### Rectification and Scrubber Water Recycle

With inclusion of a step in the present process that removes contaminants from the molecular sieve ethanol product, which is passing the ethanol through the product distillation column, an increased load of acetaldehyde and other contaminants can be processed through the rectification column without affecting the final ethanol product. A water stream containing acetaldehyde and other contaminants results from passing vapor from a fermentation vent stream through a scrubber. In one embodiment the scrubber water stream from the fermentation vapor scrubber is passed to the rectification column. This stream contains acetaldehyde and carbon dioxide from the fermenter, as well as some ethanol. In addition, the stream contains ammonia if biomass was pretreated with ammonia during preparation of hydrolysate used in the fermentation medium. The scrubber water stream enters the rectification column below the feed from the beer column, because it has a reduced level of ethanol compared to the beer column overheads, but sufficiently high up the rectification column to facilitate removal of ammonia, carbon dioxide and acetaldehyde from the bottom of the rectification column.

In addition, rather than using fresh makeup water in the fermentation vapor scrubber as is typical in a grain ethanol process, use of fresh water can be reduced in this aspect of the cellulosic ethanol process by using ethanol depleted water from the rectification column in the fermentation vapor scrubber. In one embodiment at least a portion of the ethanol depleted rectification column water stream is passed to the fermentation vapor scrubber. Thus in one embodiment ethanol depleted rectification column water and fermentation vapor scrubber water form a water recycle loop between the rectification column and fermentation vapor scrubber. These process waters (the ethanol depleted rectification column water and fermentation vapor scrubber water) may be used in the recycle loop without additional purification steps. The temperature at which water is required in the fermentation vapor scrubber is less than that of the water that exits the rectification column, so typically heat is interchanged between the water feed to the scrubber and the water returning from the scrubber to the rectification column in a process to process exchanger, with the final cooling of the water feed to the scrubber being accomplished using an exchanger where the utility stream is cooling water or chilled water.

In this recycle process, a large amount of water, which is ethanol depleted rectification column water, can be used on the scrubber to ensure more efficient capture of the acetaldehyde that enters in the fermentation vent vapor. A portion of the carbon dioxide will also be captured in the water exit stream from the scrubber, rather than being released to atmosphere as is typical. When using this recycle process, a greater amount of acetaldehyde and carbon dioxide will be present in the further ethanol-enriched rectification column vapor stream, which will pass to the molecular sieve. A majority of the acetaldehyde and carbon dioxide will pass through the molecular sieve with the product ethanol. At least a portion of the acetaldehyde and carbon dioxide are removed from the product by the product distillation column, reducing the levels to levels that are comparable to those present in a grain ethanol product, or lower. Contaminant levels in the product are typically as described above.

Cellulosic Ethanol Process with Acetaldehyde Handling

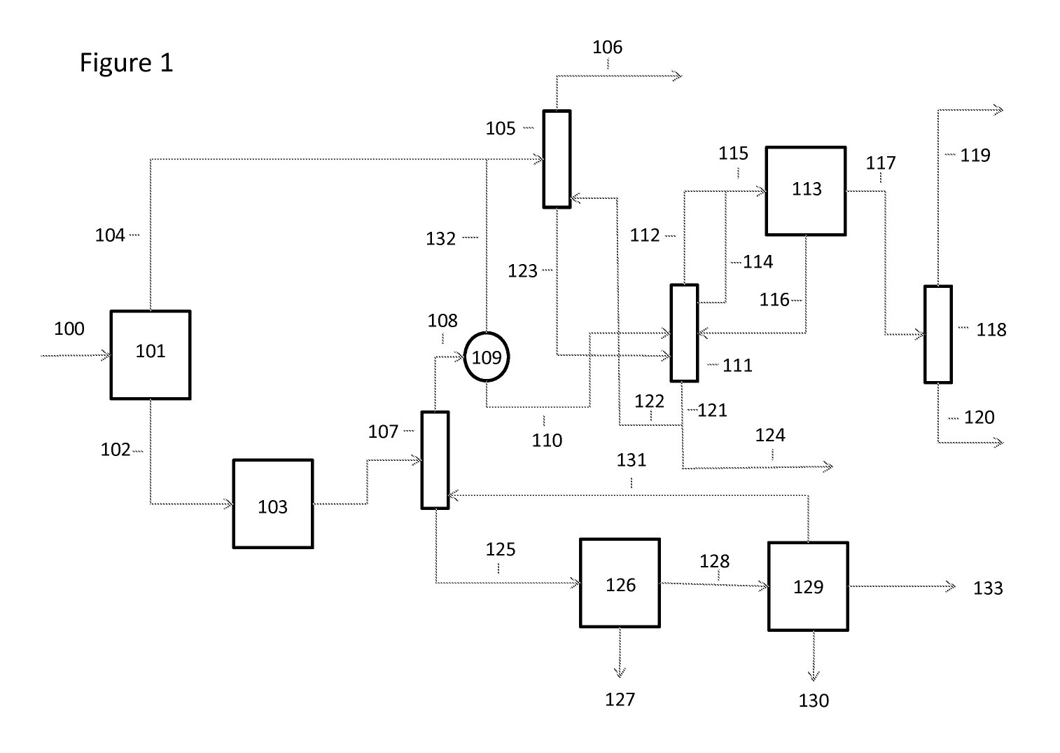

A schematic diagram in FIG. 1 shows a flow sheet representing an embodiment of process stages of the present process from the entry of feed (100) into the fermenter (101) through production of the bottoms stream (120) which is the final ethanol product from the product column (118). The feed to the fermenter includes fermentation medium containing cellulosic biomass hydrolysate and biocatalyst inoculum, which are either mixed or added separately to the fermenter.

With reference to FIG. 1, a beer flow (102) from the fermenter (101) passes to an interim storage vessel, the beer well (103). Vent gases given off in fermentation, which are principally carbon dioxide (CO₂), form a vent gas flow (104) which passes to a fermentation vapor scrubber, also called a CO₂ scrubber, (105) for recovery of ethanol and acetaldehyde. A CO₂ vent stream (106) passes to the atmosphere. Beer from the beer well is passed to a beer column (107) where ethanol with water from the beer is removed in a vapor overheads product (108; a beer column ethanol-rich vapor stream), with the remainder of the beer forming a liquid and solid stream, called whole stillage, (125) that is substantially free of ethanol.

The beer column vapor overheads product flow (108) passes to a beer column condenser (109) producing a small vent stream (132) which passes to the fermentation scrubber (105) and a liquid overheads stream (110). The resulting beer column liquid overheads product condensate flow (a beer column ethanol-rich liquid stream) (110) feeds a rectification column (111). In the rectification column there is further distillation and a rectification column overheads flow (a further ethanol-enriched rectification column vapor stream) (112) is superheated and passes to a molecular sieve unit (113) to further remove water from the ethanol stream. Also a side stream vapor product flow (114) which contains fusel oils is taken from an appropriate location of the rectification column, is combined with the rectification column overheads flow, the mixture (115) is superheated, and is passed to the molecular sieve unit (113) to further remove water from the ethanol stream. Thus fusel oils are combined with ethanol in the eventual product from the process. A molecular sieve purge (116) flows from the molecular sieve to the rectification column. This stream may instead flow to the beer column or the beer well. A dry ethanol flow (a molecular sieve ethanol product stream) (117) from the molecular sieve is passed to a product distillation column (118) where contaminants such as acetaldehyde, ammonia, and carbon dioxide are removed in a purge stream (119) and the bottoms stream (120) is the final ethanol product.

From the rectification column bottom an ethanol depleted rectification column water stream (121) exits, and a portion of this stream (122) is cooled and passed to the fermentation vapor scrubber (105) as scrubbing water. This water absorbs ethanol and acetaldehyde in the scrubber. The fermentation vapor scrubber bottom stream flow (a scrubber water stream) (123) is returned with appropriate heat interchange as a second feed to the rectification column (111) thereby recovering ethanol and acetaldehyde for further processing. Thus there is a recycle loop of water streams between the fermentation vapor scrubber and the rectification column.

The remaining ethanol depleted rectification column water stream (124) is typically heat interchanged with the feed to the rectification column (111) and is then passed for further treatment using an anaerobic digester or other purification method before being recycled in the process as process water.

The whole stillage (125) is further processed by a solids removal mechanism such as a filtration unit (126) to remove solids producing a filter cake (127). The separated liquid flows as thin stillage (128) to an evaporation train (129) and the final evaporate condensate (130) is treated and used as clean recycle water in the cellulosic ethanol production process. A syrup stream (133) containing low volatility dissolved material is also produced from the evaporator train. Treating of the evaporate is to remove high-volatility solutes and may be by any known method such as anaerobic digestion, aerobic digestion, membrane separations, including nanofiltration, ultrafiltration and/or reverse osmosis separately or integral to aforementioned biotreatment alternatives, and ion exchange separation. In this depiction the beer column (107) is heat integrated with the evaporation train by injection of steam (131) from the evaporators.

The management of acetaldehyde was demonstrated herein in Example 1 by ASPEN modeling (Aspen Plus®; Aspen Technology, Inc., Burlington, Mass.) of flow rates of relevant key components in the cellulosic ethanol process. Mass balances in the process with respect to the FIG. 1 flow diagram are shown in Table 1. With an input of 42.3 kg/hr input of acetaldehyde in the feed entering the fermenter (100) from hydrolysate medium, the majority is purged at a rate of 38.2 kg/hr in the vent stream (purge stream 119) from the product distillation column (118). A lesser amount of 3.8 kg/hr leaves with the final ethanol product stream, which is the bottoms stream (120) from the product distillation column. This amount of acetaldehyde is well within normal operating parameters for corn based processes. Only 0.3 kg/hr is allowed to leave to atmosphere via the CO₂ purge from the process, in the CO₂ vent stream (106).

### Distillation and Water Handling System

In a cellulosic ethanol process the large water load may be managed using an evaporation train, comprising multiple evaporators in excess of three or four or more evaporation effect equivalents. The evaporation train, beer column, rectification column, and solids removal mechanism may together form a distillation and water handling system with each element in the system connected to at least one other element in the system as shown in FIG. 1. The beer column, the rectification column, and the evaporation train may form a heat integrated system to optimize energy usage.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1

### ASPEN Model: Combined Cellulosic Ethanol Flowsheet

Processes described herein were demonstrated using a computational model of a process based on the flow diagram of FIG. 1. Process modeling is an established methodology used by engineers to simulate complex chemical processes. The commercial modeling software Aspen Plus® (Aspen Technology, Inc., Burlington, Mass.) was used in conjunction with physical property databases, such as DIPPR, available from the American Institute of Chemical Engineers, Inc. (New York, N.Y.) to develop an ASPEN model of an integrated ethanol fermentation, purification, and water management process.

The input stream for the model was based on the composition of the broth in the fermenter. The acetaldehyde, water, acetic acid, dissolved solids, and solids come from the biomass hydrolysate used in the fermentation medium. Ethanol and CO₂ are produced during the fermentation. The compositions of pertinent components in the streams as numbered in FIG. 1 are given in Table 1.

TABLE 1
Mass flow in kg/hr of components of a cellulosic ethanol process in streams from FIG. 1 obtained from ASPEN
modeling

| | | | | | | | dissolved | |
|---|---|---|---|---|---|---|---|---|
| stream | ethanol | water | acetaldehyde | CO₂ | fusels | Acetic acid | solids | solids |
| Input | 12729.96 | 127325 | 42.29485 | 18535.5035 | 10 | 1039.7625 | 1677.2699 | 3484.114 |
| stream | | | | | | | | |
| 102 | 12417.64 | 126979 | 36.51442 | 220.5738 | 9.719241 | 1038.957 | 1038.957 | 3483.926 |
| 104 | 312.3196 | 345.8794 | 5.780424 | 18314.93 | 0.2807593 | 0.805508 | 8.59E−18 | 0.1881033 |
| 106 | 1.223365 | 428.1066 | 0.2841412 | 18463.81 | 0.00300308 | 1.545166 | 0 | 0.0105117 |
| 132 | 238.3075 | 70.81485 | 2.091361 | 208.0867 | 0.1314376 | 0.1059609 | 0 | 0.00152048 |
| 110 | 12162.49 | 14548.01 | 34.42297 | 12.48715 | 9.587171 | 31.15997 | 0 | 15.74124 |
| 112 | 13790.02 | 1057.856 | 44.19629 | 71.69138 | 6.594535 | 3.1033E−14 | 0 | 4.1941E−41 |
| 114 | 35.61625 | 58.86732 | 0.0143949 | 0.000387467 | 5.160395 | 0.1714256 | 0 | 0.0263094 |
| 116 | 1116.666 | 1094.333 | 2.210534 | 0 | 1.763239 | 0.1714238 | 0 | 0.026309 |
| 117 | 12708.92 | 22.33447 | 42.00015 | 71.69177 | 9.99169 | 0 | 0 | 0 |
| 119 | 3.259393 | 0.00660622 | 38.17288 | 71.69177 | 1.57637E−05 | 0 | 0 | 0 |
| 120 | 12705.66 | 22.32787 | 3.827266 | 1.3798E−19 | 9.991674 | 0 | 0 | 0 |
| 122 | 10 | 49800.48 | 0.0359139 | 1.7626E−13 | 0.0160396 | 104.7544 | 0 | 54.65101 |
| 123 | 559.4037 | 49789.06 | 7.623557 | 59.20462 | 0.4252335 | 104.1248 | 0 | 54.83764 |
| 124 | 2.914468 | 14514.19 | 0.0104669 | 5.137E−14 | 0.00467466 | 30.53035 | 0 | 15.92786 |
| 125 | 16.87314 | 136358 | 8.77447E−05 | 8.9495E−29 | 0.000632648 | 1031.1257 | 1677.2699 | 3853.676 |
| 131 | 0.0259411 | 23997.36 | 5.23392E−08 | 0 | 2.90162E−07 | 23.43464 | 4.56974E−09 | 385.493 |

What is claimed is:

1. A process for the production of ethanol comprising:
a) providing medium comprising hydrolysate prepared from cellulosic biomass;
b) fermenting the medium in a fermenter in the presence of a microorganism that produces ethanol to produce a beer comprising ethanol;
c) passing the beer into a beer column where a beer column ethanol-rich vapor stream is produced;
d) condensing the beer column ethanol-rich vapor stream forming a beer column ethanol-rich liquid stream;
e) passing the beer column ethanol-rich liquid stream into a rectification column where an ethanol-enriched rectification column vapor stream and an ethanol depleted rectification column water stream are produced;
f) passing the ethanol-enriched rectification column vapor stream through a molecular sieve where a molecular sieve ethanol product stream is produced; and
g) passing the molecular sieve ethanol product stream through a product distillation column where a final ethanol product and a contaminant stream are produced.

2. The process of claim 1 wherein the hydrolysate is produced by a process comprising the treatment of lignocellulosic biomass with mid ammonia.

3. The process of claim 1 or 2 wherein the final ethanol product contains less than about 800 ppm of acetaldehyde.

4. The process of claim 3 wherein the final ethanol product contains less than about 700 ppm of acetaldehyde.

5. The process of claim 3 wherein the final ethanol product contains less than 10 ppm of CO₂.

6. The process of claim 3 wherein the final ethanol product contains less than 1 ppm of ammonia.

7. The process of claim 1 or 2 further comprising:
h) passing at least a portion of the ethanol depleted rectification column water stream of (e) to a fermentation vapor scrubber that is connected to a vent stream from the fermenter, producing a scrubber water stream comprising water, ethanol, acetaldehyde, and carbon dioxide; and
i) passing the scrubber water stream to the rectification column;
wherein the ethanol depleted rectification column water and fermentation vapor scrubber water form a water recycle loop between the rectification column and fermentation vapor scrubber.

8. The process of claim 7 wherein the water recycle loop comprises ethanol depleted rectification column water and fermentation vapor scrubber water without additional purification steps.

9. The process of claim 7 further comprising passing the molecular sieve ethanol product stream of (f) through a condenser or partial condenser prior to passing it through the product distillation column of (g).

10. The process of claim 7 further comprising treating the contaminant stream of (g) using a unit selected from a boiler, a catalytic converter, a catalytic oxidizer, a thermal oxidizer, and any combination thereof.

11. The process of claim 1 wherein the cellulosic biomass is selected from the group consisting of corn cobs, crop residues such as corn husks, corn stover, grasses, wheat straw, barley straw, hay, rice straw, switchgrass, waste paper, sugar cane bagasse, sorghum plant material, soybean plant material, components obtained from milling of grains, trees, branches, roots, leaves, wood chips, sawdust, shrubs and bushes, vegetables, fruits, and flowers.

12. The process of claim 1 wherein the microorganism is an ethanologen selected from the group consisting of a yeast and a bacterium.

13. The process of claim 12 wherein the ethanologen is selected from the group consisting of Saccharomyces, Zymomonas, and Zymobacter.

14. The process of either of claim 1 wherein a portion of the ethanol depleted rectification column water is recycled to the rectification column or treated by anaerobic digestion.

15. The process of either of claim 1 wherein the ethanol-rich rectification vapor stream is mixed with fusel oils from the rectification column prior to passing through the molecular sieve.

16. The process of either of claim 1 wherein a molecular sieve purge is passed to the rectification column.

17. The process of either of claim 1 wherein whole stillage from the beer column is processed to thin stillage by filtration and condensed to syrup and a condensate by multiple evaporators.

18. The process of either of claim 17 wherein the multiple evaporators exceed three.

19. The process of claim 18 wherein the multiple evaporators exceed four

20. The process of claim 17 wherein the multiple evaporators are heat integrated with the beer column using a steam injection from the evaporators.

21. The process of claim 17 wherein the condensate is recycled.
